(19) United States
(12) Patent Application Publication
(10) Pub. No.: US 2007/0259004 A1
(43) Pub. Date: Nov. 8, 2007

(54) Expression vector for Bacillus species

(75) Inventor: Wolfgang Schumann, Bayreuth (DE)

Correspondence Address: PROSKAUER ROSE LLP, 1001 PENNSYLVANIA AVE, N.W.,, SUITE 400 SOUTH, WASHINGTON, DC, 20004, US

(73) Assignee: Universitaet Bayreuth, Bayreuth (DE)

(21) Appl. No.: 11/635,625

(22) Filed: Dec. 8, 2006

Related U.S. Application Data
(60) Provisional application No. 60/748,201, filed on Dec. 8, 2005.

(51) Int. Cl. A61K 39/00; C07H 21/04; C12N 1/21; C12N 15/00; C12P 1/04

(52) U.S. Cl. 424/246.1; 435/252.31; 435/320.1; 435/69.1; 536/22.1

(57) ABSTRACT

The present invention is related to the use of the gsiB promoter as an inducible promoter in an expression system, whereby the promoter can be induced by a measure selected from the group comprising decrease in pH, increase in temperature, addition of alcohol, preferably ethanol, exhaustion of nutrients and oxygen limitation.

### RELATED APPLICATIONS

This application claims the benefit of priority to U.S. provisional patent application No. 60/748,201 titled “Expression Vector for Bacillus Species” filed Dec. 8, 2005, the teachings of which are incorporated herein by reference.

### BACKGROUND

The present invention is related to the use of the gsiB promoter, a nucleic acid replicon comprising such promoter, a host cell comprising such respective nucleic acid replicon and a vaccine comprising such host cell.

High-level production of recombinant proteins is a prerequisite for their subsequent purification. In most cases production of heterologous proteins uses Escherichia coli cells as a protein factory (see recent review article (Schumann W et al., Gen. Mol. Biol. 27:442-453)). Attempts to develop Bacillus subtilis as a second protein factory where the recombinant proteins are secreted into the medium have not been successful because of two major reasons: (i) structural instability of the recombinant plasmids, and (ii) instability of the recombinant proteins due to degradation (Bron S W et al., Res. Microbiol. (1991) 142:875-883; Ehrlich S D et al., Res. Microbiol. (1991) 142:869-873; Wong S-L-, Curr. Opin. Biotechnol. (1995) 6:517-522). All convenient vector plasmids have been derived from natural plasmids detected in Staphylococcus aureus such as pUB110 (Gryczan T J, Proc. Natl. Acad. Sci. USA (1978) 75:1428-1432), pC194 (Ehrlich S D, Proc. Natl. Acad. Sci. USA (1977) 74:1680-1682) and pE194 (Gryczan T J, Proc. Natl. Acad. Sci. USA (1978) 75:1428-1432). While these vector plasmids replicate stably in B. subtilis, addition of recombinant DNA can confer structural instability. The molecular basis for this structural instability is related to their replication mode. These plasmids replicate as rolling circles producing single-stranded DNA as an intermediate, and short direct repeats within this single-stranded DNA may lead to the deletion of one of the two repeats and the intervening DNA (Bron S et al., Mol. Gen. Genet. (1991) 226:88-96). This observation led to the development of vectors carrying an expression cassette sandwiched between the two halves of a non-essential gene such as amyE (Shimotsu W et al., Gen. Mol. Biol. 27:442-453), thrC (Guérout-Fleury A M et al., Gene (1996) 180:57-61), lacA (Härtl, B et al., J. Bacteriol. (2001) 183:2696-2699), and pyrD, gltA, and sacA (Middleton R et al., Plasmid (2004) 51:238-245).

The second major problem is related to the production of extracellular proteases which recognize and degrade most heterologous proteins secreted into the medium. The problem has been largely solved by constructing B. subtilis strains carrying six (Wu, X-C et al., J. Bacteriol. (1991) 173:4952-4958) or even eight (Wu S C et al., Appl. Environ. Microbiol. (2002) 68:1102-1108) protease null mutations. An alternative to circumvent the problem of recombinant protein instability would be to identify a host species devoid of extracellular proteases.

In view of this, the problem underlying the present invention is to provide for a promoter which allows for the efficient production of a recombinant polypeptide in Bacillus species. A further problem underlying the present invention is to provide an expression vector for Bacillus species which is both structurally stable and allows for an inducible expression of recombinant polypeptides. A further problem underlying the present invention is to provide for a vaccine.

### SUMMARY

According to the present invention the problem is solved in a first aspect by the use of the gsiB promoter as an inducible promoter in an expression system, whereby the promoter can be induced by a measure selected from the group comprising decrease in pH, increase in temperature, addition of alcohol, preferably ethanol, exhaustion of nutrients and oxygen limitation.

In an embodiment the decrease in pH is a decrease in pH of the culture medium.

In a preferred embodiment the decrease in pH is from about 6.8 to about 5.8.

In an embodiment the increase in temperature is about at least 10° C., preferably from about 37° C. to about 48° C.

In an embodiment the addition of ethanol results in an ethanol level within the culture medium of about 4%.

In an embodiment the promoter comprises a sequence according to SEQ ID NO: 1.

In an embodiment the gsiB promoter is incorporated into a vector, more preferably incorporated into an expression vector.

In an embodiment the expression system is a microorganism of the genus Bacillus, preferably the microorganism is Bacillus subtilis.

In an embodiment the expression system is for the production of a polypeptide.

In an embodiment the expression system is for the use as a vaccine, preferably an oral vaccine.

According to the present invention the problem is solved in a second aspect by a nucleic acid replicon that replicates in Bacillus, for the expression of a polypeptide, whereby the replicon comprises the backbone of a plasmid selected from the group comprising pMTLBS72, pAMβ1, and pTB19, and a gsiB promoter.

In a preferred embodiment the gsiB promoter is inserted into the SacI-BamHI restriction site.

In an embodiment the replicon comprises a transcriptional terminator.

In a preferred embodiment the transcriptional terminator is selected from the group comprising the trpA transcriptional terminator, t₀ terminator of bacteriophage lambda and the t₁t₂ terminator of the rrnB operon.

In an embodiment the transcriptional terminator is inserted between the MluI and the AatII restriction site of pMTLBS72.

In an embodiment the promoter and the transcriptional terminator form an expression cassette.

In a preferred embodiment the expression cassette is inserted between a pair of restriction sites of pMTLBS72, whereby such pair of restriction sites is selected from the group comprising SacI-BamHI, SacI-XbaI, SacI-AatII, BamHI-XbaI, BamHI-AatII, and XbaI-AatII.

In an embodiment the replicon further comprises at least one of the following elements selected from the group comprising an origin, and a selection marker.

In an embodiment the replicon comprises a nucleic acid sequence coding for a polypeptide, whereby the expression of the polypeptide is under the control of the gsiB promoter.

In an embodiment the polypeptide is selected from the group comprising enzymes, pharmaceutically active polypeptides and antigens.

In a preferred embodiment the polypeptide is the LTB antigen.

In an embodiment the replicon is a vector, preferably a plasmid.

In a preferred embodiment the vector is a shuttle vector for both E. coli and B. subtilis.

According to the present invention the problem is solved in a third aspect by a host cell comprising a nucleic acid replicon according to any of the first and second aspect of the present invention.

In a preferred embodiment the host cell is selected from the genus Bacillus.

In a preferred embodiment the Bacillus is Bacillus subtilis, preferably Bacillus subtilis strain 1012 and Bacillus subtilis strain IS58.

In an embodiment the host cell is E. coli.

According to the present invention the problem is solved in a fourth aspect by a vaccine comprising a host cell according to any of the first to third aspect of the present invention, wherein the host cell is Bacillus.

In a preferred embodiment the Bacillus is Bacillus subtilis, preferably Bacillus subtilis strain 1012 and Bacillus subtilis strain IS58.

In an embodiment the vaccine is an oral vaccine.

In an embodiment the vaccine elicits a specific immune response.

In an embodiment the vaccine comprises vegetative Bacillus.

In an embodiment the vaccine comprises Bacillus spores.

In an embodiment the antigen expressed by the host cell is LTB antigen.

In an embodiment the subject is an animal and/or a human being.

In a preferred embodiment the animal is a domestic animal, preferably cattle, sheep, pigs, goats, horses, dogs, cats and birds.

In an embodiment the polypeptide expressed by the host cell is LTB and the vaccine is for the treatment of LTB associated diarrhoea.

In an embodiment the vaccine is for the treatment and/or prevention of a disease.

According to the present invention the problem is solved in a fifth aspect by a method for the production of a polypeptide comprising the steps of:
a) providing a host cell according to any of the first to fourth aspect of the present invention, whereby the host cell encodes for the polypeptide;
b) cultivating the host cell under conditions allowing for the expression of the polypeptide; and
c) harvesting the polypeptide.

According to the present invention the problem is solved in a sixth aspect by a method for providing an immune response in a subject comprising the steps of:
a) providing a vaccine according to any of the first to fifth aspect of the present invention; and
b) administering the vaccine to the subject in an amount so as to elicit an immune response.

In a preferred embodiment the subject is a human being or an animal.

The various embodiments described herein can be complimentary and can be combined or used together in a manner understood by the skilled person in view of the teachings contained herein.

### BRIEF DESCRIPTION OF THE DRAWINGS

The present invention will now be illustrated by the attached figures and examples from which additional advantages, features and embodiments may be taken.

FIG. 1 shows the gene and restriction map of plasmid pHCMC03.

FIG. 2 shows the gene and restriction map of plasmid pLDV2.

FIG. 3 shows the result of a Western Blot analysis on the inducibility of plasmid pHCMC03.

FIG. 4 shows the result of a Western Blot analysis of the expression of LTB by the recombinant B. subtilis expression pLDV2 strain.

FIG. 5A shows a diagram indicating the in vitro stability of B. subtilis expression vectors expressed as percentages of the total number of tested colonies.

FIG. 5B shows a diagram indicating the in vivo stability of B. subtilis expression vectors expressed as percentages of the total number of tested colonies.

FIG. 6 show the administration schemes used for the immunisation of mice as reported in example 4.

FIG. 7 is a diagram illustrating the immune response expressed as IgG titre after 14 days, 28 days and 42 days upon oral administration of different Bacillus subtilis based vaccines.

FIG. 8 is a diagram illustrating the immune response expressed as IgG titre after 14 days, 28 days and 42 days upon intraperitoneal administration of different Bacillus subtilis based vaccines.

FIG. 9 is a diagram indicating the efficacy of oral vaccination as described in example 4 with the indication being the SlgA titre which is the reverse of the maximum dilution yielding a positive reaction when compared with a non-reactive serum.

FIG. 10 is a diagram indicating LTB-specific serum IgG responses in mice immunized with spores or vegetative cells of Bacillus subtilis delivered via parenteral or oral routes.

FIG. 11 is a diagram indicating induction of LTB-specific serum IgG (A) and fecal IgA (B) responses elicited in mice p.o. immunized with spores or vegetative cells of B. subtilis.

FIG. 12 is a diagram indicating serum anti-LTB IgG subclass responses elicited in mice immunized with spores or vegetative cells of B. subtilis via i.p. or p.o. routes.

### DETAILED DESCRIPTION

The present invention is based on the surprising finding of the inventor that the promoter gsiB is a highly inducible promoter allowing for high level expression of recombinant polypeptides. More particularly, the gsiB promoter has surprisingly been proven to be highly inducible by the use of both physical and chemical inducers, including acid stress, temperature stress, ethanol mediated stress, and metabolic stress such as exhaustion of nutrients and oxygen limitation. Even more particularly, the present inventor has surprisingly found that when such gsiB promoter is used in a nucleic acid replicon according to the present invention, a highly inducible and structurally stable expression vector for Bacillus species is provided. Insofar the nucleic acid replicon according to the present invention circumvents the drawbacks of the expression systems used in the prior art for the expression of recombinant polypeptides by Bacillus species and in particular by Bacillus subtilis.

In a further aspect the present inventors have surprisingly found that the expression system according to the present invention is particularly useful as a vaccine. Preferably, such vaccine comprises a host cell of the Bacillus genus which comprises the nucleic acid replicon according to the present invention. Such vaccine is particular useful insofar as it provides for a high immunogenic efficacy. Without wishing to be bound by any theory, the present inventor attributes this high immunogenic efficacy to the surprising stability of the nucleic acid replicon according to the present invention. The nucleic acid replicon according to the present invention allows for a multicopy plasmid-based expression system which is, in contrast to systems known in the art, segregational and structurally stable. Also, such multicopy plasmid-based expression system provides for a high expression level in contrast to integrative vectors which are used in the prior art to circumvent the segregational and structural instability of the expression systems for Bacillus species of the prior art. Insofar, the present invention turns away from the approaches followed in the prior art and meets a long felt need.

The gsiB promoter is a promoter recognized by the alternative sigma factor σ^{B} and is expressed at a very low level under physiological conditions in Bacillus species. It is also known to be inducible by different stresses such as heat and acid stress and ethanol (Maul B et al., Mol. Gen. Genet. (1995) 248:114-120; Völker U. et al., Microbiology (1994) 140:741-752).

However, it is for the first time according to the present inventor that an inducible system for the expression of recombinant polypeptides in Bacillus species has been provided the expression level of which can be increased by the use of both physical and chemical measures, whereby both types of measures are easy to apply and do not interfere with the fermentation of the Bacillus organism and subsequent down-stream processing thus allowing for a large-scale use of such systems. Particularly preferred measures are decrease in pH and increase in temperature. It seems that it is this particular characteristic of the promoter which provides for the advantageous use of the vaccine according to the present invention and more particularly for the enhanced and stabilized production of any antigen by such vaccine under both in vivo and in vitro conditions. In other words, the combination of a recombinant protein or polypeptide under the control of the gsiB promoter and thus the expression of said recombinant protein and polypeptide, respectively, under the control of the acidic pH in a host organism, in particular the gastrointestinal tract thereof, such as either man or animal, provides for the surprisingly advantageous effect of the vaccine according to the present invention. Moreover, although not active during the sporulation phase, the gsiB promoter proved to be induced during the transit of spores into the mammalian host.

The decrease in pH refers preferably to the decrease of the pH of the culture medium where a host organism containing a nucleic acid replicon such as a plasmid vector containing the gsiB promoter as described herein, is active. Preferably, as used herein the term active means that the transcription and translation system of the host organism is capable of producing the recombinant polypeptide encoded by the nucleic acid replicon. The culture medium, as preferably used herein, is thus not necessarily a culture medium containing the nutrients required for growth and/or maintenance of the respective host organism, but is basically any liquid in which the Bacillus microorganism can be active or contained without being killed. Preferably, the liquid is a physiological saline solution. Such decrease in pH can, among others, be affected by the use of acidic metabolites of the Bacillus microorganism or by organic and inorganic acids. Alternatively, and in particular in an industrial setting, the decrease in pH suitable to trigger the expression of a nucleic acid under the control of the gsiB promoter occurs via addition of mineral acids, including but not limited to, HCl.

A preferred culture medium is LB medium or LB medium supplemented with 0.5% glucose and 1.5% mM KH₂PO₄.

A further measure which may be applied in connection with the use of the gsiB promoter according to the present invention or the nucleic acid replicon according to the present invention is an increase in temperature. Preferably, the increase in temperature is about 10° C. Given the growth temperature of mesophilic Bacillus species, an increase of temperature of the culture medium as defined herein would preferably be from about 37° C. to about 48° C. This increase in temperature can be easily realized, e.g. by adding thermal energy to the culture flask or the reactor containing the Bacillus microorganism. Such thermal energy can be delivered by heaters and other thermal energy delivery means known to the one skilled in the art.

When using the addition of ethanol to induce the promoter, this is typically done by adding ethanol to the culture medium. Preferably the ethanol is about 4% (v/v) based on the overall culture medium.

The specific induction of the gsiB promoter by using any of the aforementioned measures, in particular in connection with the nucleic acid replicon according to the present invention, provides for an advantage over measures otherwise used in connection with the induction of Bacillus expression systems.

The gsiB promoter is described, for example, in Maul B. et al., Mol Gen Genet (1995) 248:114-20; or Völker U et al., Microbiol (1994) 140:741-52. This form of the gsiB promoter will also be referred to herein as the wildtype gsiB promoter. It will be understood by the ones skilled in the art that derivatives of such wildtype gsiB promoter are also comprised by the term gsiB promoter as used herein. Such derivatives of the wildtype gsiB promoter can be derived from the gsiB promoter according to SEQ ID NO: 1 (GTTTGTTTAA AAGAATTGTG AGCGGGAATA CAACAACCAA CACCAATTAA AGGAGGAATT) by the ones skilled in the art. Such gsiB promoter derivative typically exhibits one or several mutations which may comprise insertion as well as deletion in the promoter sequence provided that such promoter derivative is still capable of being inducible by any of the measures disclosed herein and provides for an expression of a polypeptide which is under the control of such promoter. Preferably the level of expression is at least about 50% of the respective activity of the gsiB promoter according to SEQ ID NO: 1. As used herein, the term gsiB promoter and in particular the term wildtype gsiB promoter is not limited to the gsiB promoter having the specific nucleic acid sequence according to SEQ ID NO: 1 or stemming from B. subtilis or closely related genera such as Staphylococcus aureus. Rather the term comprises any gsiB promoter, preferably any gsiB promoter from a Bacillus species. As preferably used herein the term gsiB promoter is sigmaB-dependent promoter which controls the expression of the gsiB gene. The gsiB gene is preferably selected from the group comprising the gsiB gene from Bacillus subtilis, the gsiB gene from Bacillus licheniformis, the gsiB gene from Bacillus clausii and the gsiB gene from Lactobacillus sakei. Insofar, the term gsiB promoter comprises in a preferred embodiment also those promoters controlling the gsiB gene in Bacillus subtilis, Bacillus licheniformis, Bacillus clausii and Lactobacillus sakei. In a preferred embodiment the gsiB gene codes for a GsiB protein, whereby the GsiB protein preferably has the following amino acid sequence (taken from Bacillus subtilis):

MADNNKMSREEAGRKGGETTSKNHDKEFYQEIGQKGGEATSKNHDKEFYQEIGEKGG EATSKNHDKEFYQEIGEKGGEATSENHDKEFYQEIGRKGGEATSKNHDKEFYQEIGSKG GNARNND (SEQ ID NO: 8)

In a further aspect the present invention is related to a nucleic acid replicon. As used herein, a replicon is preferably a discrete unit of a nucleic acid that replicates independently of the chromosome of the bacterial cell acting as host for such replicon. Insofar, preferably the term replicon excludes the chromosome of such host bacterial cell. The replicon can be presented as a single copy or as multiple copies. In each case, the elements necessary for replication of the nucleic acid replicon are found in the nucleic acid of the replicon or can be found in the host bacterial cells containing the replicon so that the replicon can be produced within the bacterial cells from one generation to the next, or can be produced and recovered in a form that can be introduced in bacterial cells of a culture different from that in which they reproduce, to initiate further rounds of replication.

The replicon according to the present invention comprises at least the gsiB promoter and a backbone of a plasmid. The backbone is preferably provided by pMTLBS72 which is, among others, described in (Titok M A et al., Plasmid (2003) 49:53-62). It is also within the present invention that a nucleic acid replicon may comprise a gsiB promoter and a backbone of a plasmid different from pMTLBS72. Such different plasmids are, among others and not limited thereto, pAMβ1 (L. Jannière et al. (1990) Gene 87:53-61) and pTB19 (S. Bron et al. (1987) Plasmid 14:185-194).

In preferred embodiments, the nucleic acid replicon comprises, in addition to the gsiB promoter, several elements which are encoded in the order of nucleotides of the nucleic acid of the replicon and which are preferably provided by the backbone of a plasmid. These elements can be sites necessary for replication of the replicon, such as an origin of replication, having one or more sites for recognition by DNA polymerase. Other sites can include an operator region which can have one or more sites to which the repressor can bind to regulate transcription initiation at the promoter. Downstream from the promoter, i.e. during transcription in 5′ to 3′ direction with respect to the sense strand, a linker site can be included. Such linker site may comprise one or more restriction sites which are cleavable by the use of one or more restriction enzymes under conditions appropriate for restriction enzyme activity. A linker site can be positioned such that it lies outside of any desired coding sequence in which case it can be used for the insertion of a segment of a nucleic acid having a sequence encoding a gene product such as a recombinant polypeptide. In an alternative arrangement, for producing a fusion polypeptide, a linker site can be positioned such that it lies within or adjacent to a first sequence encoding a first polypeptide which is sometimes called a “carrier” protein or polypeptide, such that insertion into the linker site of a segment of nucleic acid having a second sequence encoding a second polypeptide results in a gene which encodes, under the regulation of the promoter/operator region, a fusion polypeptide having an amino acid sequence encoded, in part, by the first nucleic acid, and, in part, by the second nucleic acid sequence.

It will be acknowledged that preferably, the nucleic acid replicon is a vector, and more preferably a plasmid vector. The nucleic acid replicon is in a further embodiment a so-called shuttle vector which allows for the replication of the nucleic acid replicon in two microorganism species, whereby, preferably, the two microorganism species are selected from different genera. Preferably, the shuttle vector allows for the replication of the nucleic acid replicon according to the present invention in both Bacillus, preferably Bacillus subtilis, and E. coli.

A further element which is typically contained in the nucleic acid replicon is a transcriptional terminator. Such transcriptional terminator allows for the production of a nucleic acid and, in case such nucleic acid is a coding nucleic acid, of a polypeptide having a defined C terminal end. It will be acknowledged that it is particularly advantageous to combine the promoter, e.g. the gsiB promoter, and the transcriptional terminator so as to create an expression cassette. Such expression cassette can easily be incorporated into a backbone of a plasmid, such as pMTLBS72. Even more preferably, such expression cassette contains a linker site between the promoter and, if present, the operator, and the transcriptional terminator.

In principal, the transcriptional terminator can be any transcriptional terminator which is recognized and active in Bacillus. Particularly preferred terminators are the trpA terminator, the to terminator of the bacteriophage lambda and the t₁t₂ terminator of the rrnB operon of E. coli.

The particular embodiment of the nucleic acid replicon which is referred to herein as pHCM03, such expression cassette comprising the gsiB promoter and the trpA terminator is inserted between the MluI and AatII restriction sites of pMTLBS72. Other sites into which any terminator can be cloned, are between the SacI and XbaI restriction sites, the SacI and AatII restriction sites, the BamHI and XbaI restriction sites, the BamHI and AatII restriction sites, and the XbaI-AatII restriction sites.

Downstream of the gsiB promoter a nucleic acid coding for a polypeptide is inserted in a preferred embodiment. The expression of the polypeptide is under the control of the expression cassette or part of such expression cassette. It will be acknowledged that, in principle, any polypeptide can be under the control of said expression cassette and thus under the control of the gsiB promoter.

As used herein, the term polypeptide preferably means any polymer consisting of two or more amino acid residues linked through a peptide bond. Preferably the amino acids are proteinaceous D-α-amino acids. Accordingly, a polypeptide can be as short as comprising two amino acid residues only and as long as comprising several hundreds of amino acid residues which may also be referred to, particularly in the art, as protein rather than polypeptide. Also, the term polypeptide comprises both post-translationally modified as well as non-post-translationally modified polypeptides. A post-translational modification may be, among others, phosphorylation, acetylation and glycosylation. The polypeptide the expression of which can be controlled by the gsiB promoter in such nucleic acid replicon can belong to any of the groups of enzymes, pharmaceutically active polypeptides and antigens. It will be acknowledged that the aforementioned generic terms may overlap to a certain extent. Preferably, the enzymes are enzymes which are relevant for industrial application and particularly comprise proteases and amylases. Pharmaceutically active polypeptides may comprise hormone and hormone-like factors as well as growth factors and cytokines. Also, the polypeptide can be an antigen or group of antigens. As preferably used herein, an antigen is any polypeptide which is suitable to elicit an immune response in an organism confronted with such antigen, whereby the organism is preferably a mammalian organism or a bird.

It will be acknowledged by the ones skilled in the art that the antigen the expression of which is under the control of the gsiB promoter is preferably an antigen the immune response against which is suitable to confer protection against a disease which involves such antigen. Such protection can be generated prior to a subject suffering from such disease, e.g. for prevention purpose, as well as when a subject is already suffering from such disease, e.g. for treatment purpose. It will also be acknowledged that in principle, any antigen can be expressed by the expression system according to the present invention and the vaccine according to the present invention, respectively. The respective sequence which has to be cloned into the expression system and replicon, respectively, can either be retrieved from any databanks or can be determined by routine sequence analysis. It is also within the present invention that the sequence may be modified. Such modification comprises in a preferred embodiment a measure which is selected from the group comprising truncation, extension, change or alteration of single or multiple amino acids and adaptation to the codon usage of the host organism. It has been found by the present inventors that the polypeptide expressed by the replicon according to the present invention can be detected both in the exponential phase cells incubated at elevated temperatures and in stationary phase cells deprived of nutrients.

A preferred antigen is the LTB antigen which is the e subunit of the heat labile toxin B from enterotoxic E. coli strain (ETEC). Other antigens which are useful in or for the practice of the present invention are known by the ones skilled in the art and will also comprise antigens which will be discovered in the future. Due to the underlying mechanism the ones skilled in the art will also know how and for which disease also such future antigens will be used. A further particularly preferred antigen is one selected or derived from the virus causing bird flu, whereby the disease for which a respective vaccine according to the present invention, which comprises such antigen, can be used, is bird flu.

In a further aspect the present invention is related to a host cell comprising a nucleic acid replicon according to the present invention.

A host cell as used herein particularly refers to a cell that has the capacity to act as a host and expression vehicle for an incoming sequence, e.g. a sequence introduced into the cell, as described herein. Preferably such sequence is the nucleic acid replicon and the nucleic acid coding for a recombinant polypeptide, respectively. In a preferred embodiment the host cell is a microorganism. In a more preferred embodiment the host cells are Bacillus species. In a particularly preferred embodiment, the term Bacillus refers to all species, subspecies, strains and other taxonomic groups within the genus Bacillus, including, but not limited to B. subtilis, B. licheniformis, B. lentus, B. brevis, B. stearothermophilus, B. alcalophilus, B. amyloliquefaciens, B. coagulans, B. ciruclans, B. lautus and B. thuringensis. Particularly preferred is B. subtilis, and more particularly strain 1012 of Bacillus subtilis, strain IS58 of Bacillus subtilis, strain 168 of B. subtilis and derivatives of B. subtilis strain 168.

However, in the embodiment where the nucleic acid replicon is a shuttle vector, the host organism is not limited to a single host organism but also comprises a second host organism. Preferably, such second host organism is selected from the group Enterobacteriaceae and more preferably E. coli.

In a further aspect the present invention is related to a vaccine comprising a host cell according to the present invention, whereby the host cell is a host cell of a Bacillus species. It will be acknowledged that the Bacillus species is preferably any of the Bacillus species known to the ones skilled in the art and more particularly the Bacillus species and strains disclosed herein. Particularly preferred is Bacillus subtilis, and most preferably Bacillus subtilis strain 1012, Bacillus subtilis strain IS58 Bacillus subtilis strain 168 and derivatives of Bacillus subtilis strain 168.

As used herein a vaccine is an agent which is, upon administration to a subject, suitable to elicit an immune response in said subject. Preferably, the immune response is such as to provide protection against a disease associated with the antigen, or allows for the treatment of such disease. In a preferred embodiment the vaccine is an oral vaccine.

The vaccine, as preferably used herein, is present in or as a pharmaceutical formulation known to the ones skilled in the art. Typically, the vaccine comprises as such or separate therefrom, one or several adjuvants. Appropriate adjuvants are known to the one skilled in the art and comprise, but are not limited to, complete Freund's adjuvant, incomplete Freund's adjuvant, Al₂O₃, GroEL of Mycobacterium bovis, and listeriolysin O of Listeria monocytogenes.

It will be acknowledged that the vaccine according to the present invention may be present in a solid form, preferably powder form, or in a liquid form. The vaccine is more preferably present in a lyophilised form. In a preferred embodiment, the vaccine comprises spores rather than live microorganisms, in particular if the vaccine is for oral administration. Irrespective of the route of administration, a spore based vaccine according to the present invention goes along with advantages over vaccines which are based on live microorganisms. Such advantages are related to a prolonged storability because spores may be stored for decade and can be suspended in drinking water without germination, and to an easy use even by less trained people.

The vaccine is preferably administered to a subject in need thereof in a liquid form. This is preferably achieved by suspending the host cell in a liquid. Preferably, the liquid is a water-based buffer, more preferably a physiological buffer or a suspension. The vaccine may be administered by, among others, injection or by inhalation. In case the vaccine is to be administered by inhalation, the present invention also comprises respective aerosols containing such vaccine. It is known to the one skilled in the art how to prepare such aerosols.

In case the vaccine is administered by injection, the injection can use various routes of administration known to the one skilled in the art, comprising, but not limited to, intravenous, intramuscular, intradermal, subcutaneous, oral and parenteral and the like.

In an embodiment, the vaccine comprises live Bacillus host cells of one or several Bacillus species. Alternatively, the vaccine comprises spores. It is also within the present invention that both life Bacillus microorganisms as well as spores of Bacillus are administered as a vaccine.

Without wishing to be bound by any theory, it seems that the vaccine according to the present invention allows for the efficient expression of an antigen, whereby the expression of the recombinant polypeptide occurring in and by the host organism forming the vaccine according to the present invention which is in a preferred embodiment an oral vaccine, is activated or induced by the passage through the stomach or by phagosomes of antigen presenting cells providing for an acid shock, an increased temperature and anaerobic environments which allow for the induction of the gsiB promoter. This may occur both at the level of the vaccine comprising live microorganisms of Bacillus as well as at the level of the vaccine comprising spores thereof. Again without wishing to be bound by any theory, the embodiment of the vaccine according to the present invention which predominantly or solely comprises spores rather than live microorganisms, is effective as the spores are stable in the gastrointestinal tract of the animals to be treated. Some of the spores are taken up by the M cells of the intestine and passed on to phagocytic cells. The spores germinate in the phagosomes and express the antigen under the control of the gsiB promoter. The increased efficacy of the vaccine according to the present invention allows for a reduced immunisation protocol in terms of reduced stimulations compared to the Bacillus based vaccine of the prior art. Such reduction can be down to one third of those previously applied in immunization regimens employing recombinant spores genetically modified to express heterologuous antigens genetically fused to spore coat proteins expressed only during the sporulation phase. Additionally and assumingly because of this, the vaccine according to the present invention allows to avoid the generation of tolerance by the organism subject treated with the vaccine according to the present invention.

It will be acknowledged by the one skilled in the art that using the vaccine according to the present invention, a specific immune response can be generated. More particularly such immune response is a specific immune response to the polypeptide and more particularly to the antigen expressed by the replicon according to the present invention. The immune response elicited by the vaccine according to the present invention preferably results in the generation of IgG antibodies directed against the antigen and IgA antibodies against the antigen. Within the IgG antibodies, preferably IgG2a and IgG1 are predominant. It has been found that no IgE is induced.

It will be acknowledged by the ones skilled in the art that the immunization regimen as well as the immune response obtained will depend on whether vegetative Bacillus cells or spores of Bacillus are used. Using spores a significant systemic and secreted antibody response can be obtained either after a single i.p. dose or a single set of three consecutive daily p.o. doses. This observation confirms that recombinant spores can elicit specific antibody responses to the vaccine antigen assumingly by the spores germinating during their transit through the gastrointestinal tract and inside phagocytic cells. Taking into account that no vaccine antigen is carried by the vaccine spores, the specific immune response elicited can be solely attributed to in vivo synthezised antigens. The higher IgG2a/IgG1 subclass ratios indicate that the specific responses are activated under in vivo conditions, most probably by antigen expression after germination of phagocytosed spores both at the mucosal and systemic immune response afferent sites.

Using vegetative Bacillus, more doses are needed compared to the immunization regimen using Bacillus spores. The reason therefore seems to be the massive cell death occurring during the transit through the gastrointestinal tract. The more balanced IgG2a/IgG1 subclass response elicited also indicates that in vivo gene expression does note occur after phagocytosis of vegetative cells by antigen presenting cells. According to the current understanding of the present inventor, Bacillus cells operate by simply protecting the expressed antigen during the transit through the gastrointestinal tract allowing for interaction with M cells at Peyer's patches as inert vehicles.

It is within the present invention that actually any antigen can be expressed by the host organism according to the present invention. Thus, the vaccine according to the present invention is in principle suitable for both prevention and/or treatment of any disease which involves any antigen which is or can be expressed by the nucleic acid replicon according to the present invention.

A particular preferred antigen is the LTB antigen and the respective disease for the prevention and/or treatment of which the vaccine according to the present invention may be used, is diarrhoea.

In a further aspect the invention is related to the production of a recombinant polypeptide. Such method comprises the provision of a host cell according to the present invention, whereby the host cell encodes for the recombinant polypeptide the production of which is intended. Subsequently, the host cell is cultivated in a culture medium under conditions allowing for the expression of the polypeptide. Preferred cultivation conditions for the individual Bacillus species are known to the ones skilled in the art. Depending on the particular Bacillus species used, it is possible that the expression of the polypeptide goes along with growth. Alternatively, the expression of the polypeptide may be induced at or after a certain stage of growth, preferably at the stationary phase. Means to determine whether a culture is actually at a stage appropriate for induction of the expression of the recombinant protein, are known to the one skilled in the art. A preferred cultivation method used in connection with the method according to the present invention is a batch fermentation, although it will be acknowledged that also fed-batch, repeated batch or continuous fermentation are suitable means for the cultivation of the host organism. In a further step the method for the production of the polypeptide comprises the step of harvesting the polypeptide.

The process of harvesting a polypeptide is known by the one skilled in the art. It is within the present invention that the culture medium as such is already used in accordance with the present invention. The inventive method also comprises the step of separating the cells from the culture medium as a part of the harvesting step. In a further aspect, the harvesting also involves the isolation and/or purification of the respective polypeptide. Means to isolate and/or purify the polypeptide are known to the one skilled in the art. Respective methods include, however, are not limited thereto, concentration, filtration and chromatography. It will be acknowledged by the one skilled in the art that the applicable harvesting and in particular the isolation and purification scheme(s) strongly depend on the individual polypeptide and are principally known by the one skilled in the art.

In a further aspect that invention is related to a method for providing an immune response in a subject by administering to said subject the vaccine of the present invention. Preferably, the subject is a human being or an animal. Preferably the animal is a mammal and more preferably the mammal is a domestic mammal.

It will be acknowledged that such method can be applied to a subject in need of such treatment, either for prevention of a disease or treatment of a disease. It will also be acknowledged by the ones skilled in the art that the regimen for the treatment of a disease depends on the particular disease to be treated or prevented and appropriate vaccination regimens are known to the one skilled in the art and can preferably be deduced from the results disclosed herein for the immunization of mice.

### EXAMPLE 1

### Design of Plasmid pHCMC03

Plasmid pHCMC03 was basically designed by incorporating the gsiB promoter into the E. coli-B. subtilis shuttle vector pMTLBS72 (Titok M A et al., Plasmid (2003) 49:53-62) into which the trpA transcriptional terminator was introduced between the MluI and AatII restriction sites to ensure efficient termination of transcription immediately downstream of the recombinant genes by using the two complementary oligonucleotides (ON1 and ON2) (SEQ. ID: NO: 2 and SEQ. ID NO: 3, respectively) described in Kaltwasser M T et al., Appl. Environ. Micorbiol. (2002) 68:2624-2628). The sequences of the respective complementary oligonucleotides read as follows:

| | |
|---|---|
| ON1: | GGCCATGGATCCTCACTCCTACTATTAAACGCAAAATAC |
| ON2: | GGCCATGGATCCTCACTCCTACTATTAAACGCAAAATAC |

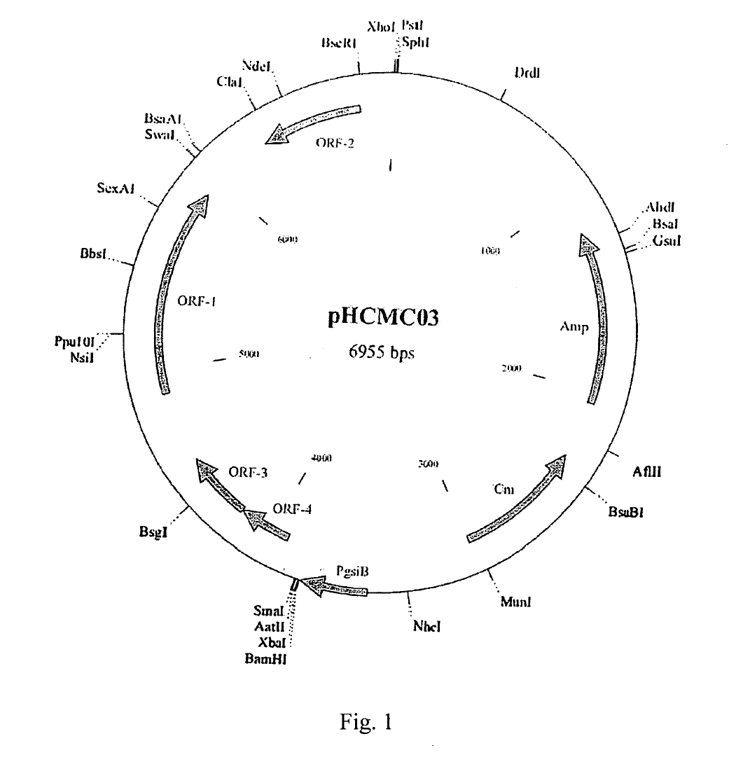

Into the thus generated backbone the gsiB promoter was inserted using the complementary oligonucleotides ON3 (SEQ ID NO: 4) and ON4 (SEQ ID NO: 5). The thus generated plasmid vector which is also replicating in both E. coli and B. subtilis, is referred to as pHCMC03 and depicted in FIG. 1.

### EXAMPLE 2

### Design of Plasmid pLDV2

A further nucleic acid replicon according to the present invention is the plasmid pLDV2 which was generated as follows.

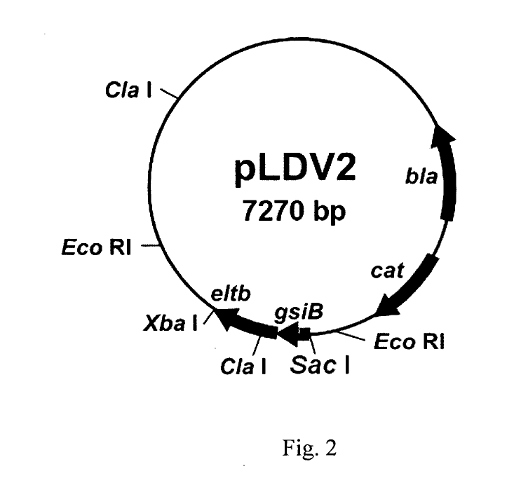

The gsiB gene upstream region encompassing the promoter and ribosome-binding site was amplified with primers ON3 (5′ GGC CAT GGA TCC_CTA TCG AGA CAC GTT TGG CTG 3′) (SEQ ID NO: 4) and ON4 (5′ GGC CAT GAG CTC TTC CTC CTT TAA TTG GTG TTG GT 3′, restriction sites underlined) (SEQ ID NO: 5) and cloned into SacI-BamHI double-digested pMTLBS72 (Titok M A et al.; Plasmid (2003) 49:53-62). After restriction analysis, one clone containing the insert was chosen and the recombinant plasmid pLMTLgsiB isolated for a final cloning step with an amplified fragment containing the elTB gene derived from ETEC H10407 strain (Evans D G; Infect Immun (1975) 12:657-67). Amplification of the elfB was carried out with primers ELTBFw (5′ TCT ATG TAG ATC TAT GGC TCC TCA GTC TAT TAC AGA 3′) (SEQ ID NO: 6) and ELTB2Rv (5′ TTT TAA TTC TAG ATT AGT TTT CCA TAC TGA TTG CCG C 3′) (SEQ ID NO: 7). The amplified fragment was force cloned into the BamHI and XbaI sites of pLDV1 originating in the final vector pLDV2 (FIG. 2). The correct cloning of the eltB gene was confirmed both by restriction analysis and nucleotide sequencing.

### EXAMPLE 3

### Cultivation of Microorganisms

The B. subtilis WW02 strain (leuA8 metB5 trpC2 hsrdRMl amyE::neo) was used for all immunization experiments (Wehrl W et al.; J Bacteriol (2000) 182:3879-73). The B. subtilis LDV1 and LDV2 and LDV3 strains were obtained after transformation with the pLDV1, pLDV2 and pREP9 (Le Grice S F J et al.; In Goeddel D V editor. Methods in Enzymology: Academic Press (1990) 185:201-14) expression vectors, respectively. All manipulations involved in vector construction and cloning of the LTB-encoding gene were performed with the E. coli strain DH5α as a recipient. Bacterial strains were routinely grown in Luria-broth (LB), and plates were prepared with added neomycin (25 μg/ml) and/or chloramphenicol (5 μg/ml), for B. subtilis, or ampicillin (100 μg/ml) for E. coli. Sporulation of the B. subtilis strains was performed in DSM (Difco-sporulation media) using the exhaustion method as previously described (Nicholson W L; in Harwood C R, Cutting S M editors. Molecular biological methods for Bacillus. Chichester, UK: Wiley (1990) 391-450). E. coli competent cells were prepared with the CaCl₂-mediated transformation protocol, while B. subtilis cells were submitted to the two-step transformation method, previously described (Sambrook J et al.; Molecular cloning: a laboratory manual. 2nd ed. Cold Spring Harbor, N.Y.: Cold Spring Harbor Laboratory (1989) Cutting S M et al.; in Harwood C R, Cutting S M editors. Molecular biological methods for bacillus. Chichester, UK: Wiley (1990) 27-74).

Preparation of Spores

Sporulation of either wild-type or recombinant strain was induced in DSM using the exhaustion method as described in a previous study (Nicholson W L; in Harwood C R, Cutting S M editors. Molecular biological methods for Bacillus. Chichester, UK: Wiley (1990) 391-450). Sporulating cultures were harvested 24 h after initiation of sporulation, subjected to lysozyme treatment to break any residual sporangial cells, followed by successive washes in 1 M NaCl and 1 M KCl and then twice in water. PMSF (10 mM) was included in washes to inhibit proteolysis. Finally, the spores were suspended in water and treated at 68° C. for 1 h to inactivate any residual cells. Viable spores were titrated for determination of the number of CFU/ml and then transferred to −20° C. until use.

### EXAMPLE 4

### Expression Characteristics of Plasmid pHCMC03 Expressing lacZ and bgaB

In order to test the inducibility and expression level of plasmid pHCMC03, the lacZ and bgaB gene, respectively, were put under the control of the gsiB promoter. This resulted in plasmids pHCMC03-lacZ and pHCMC03-bgaB. These plasmids were introduced into both B. subtilis strains 1012 and IS58. Respective cultures thereof were grown to the mid-exponential growth phase and then treated with various factors in order to induce the pgsiB promoter, namely acid stress by decreasing the pH of the medium from 6.8 to 5.8, heat stress by increasing the temperature from 37° C. to 48° C., and addition of ethanol to a final concentration of 4%.

The increase in enzyme activity of both lacZ and bgaB was measured and the results are depicted in table 1.

| | | | | Recipient | Time [min] after induction | | | | | |
|---|---|---|---|---|---|---|---|---|---|---|
| Plasmid | Promoter | Inducer | Reporter gene | strain | 0 | 5 | 10 | 20 | 30 | 60 |
| pHCMC02 | P_{lepA} | — | bgaB | 1012 | 2.4 ± 0.9 | 2.6 ± 0.5 | 4.1 ± 1.8 | 3.6 ± 1.3 | 1.6 ± 0.2 | 1.5 ± 0.4 |
| pHCMC03 | P_{gsiB} | acid | lacZ | 1012 | 19.8 ± 3.5 | 19.2 ± 3.7 | 39.3 ± 5.4 | 74.4 ± 5.5 | 188 ± 63 | 255 ± 48 |
| | | shock | | | | | | | | |
| pHCMC03 | P_{gsiB} | acid | lacZ | 1S58 | 7.7 ± 0.3 | 11.1 ± 2.0 | 24.3 ± 2.6 | 33.6 ± 4.2 | 32.5 ± 3.0 | 56.5 ± 5.2 |
| | | shock | | | | | | | | |
| pHCMC03 | P_{gsiB} | ethanol | bgaB | 1012 | 12.0 ± 5.3 | 15.7 ± 3.4 | 19.1 ± 5.9 | 32.2 ± 8.2 | 20.9 ± 1.7 | 32.8 ± 1.3 |
| pHCMC03 | P_{gsiB} | ethanol | bgaB | 1S58 | 3.1 ± 0.5 | 20.8 ± 5.1 | 19.2 ± 3.7 | 30.6 ± 7.5 | 32.8 ± 9.2 | 36.7 ± 5.7 |
| pHGMC03 | P_{gsiB} | heat | bgaB | 1012 | 12 ± 5.3 | 13 ± 1.4 | 21.9 ± 3.4 | 23.7 ± 10.2 | 20.4 ± 3.0 | 33.7 ± 1.1 |
| | | shock | | | | | | | | |
| pHCMC03 | P_{gsiB} | heat | bgaB | 1S58 | 3.1 ± 0.5 | 18.6 ± 9.0 | 22.8 ± 6.8 | 20.3 ± 3.2 | 19.1 ± 2.1 | 23.4 ± 3.9 |
| | | shock | | | | | | | | |
| pHCMC04 | P_{xylA} | 0.5% | bgaB | 1012 | 0.0 ± 0.0 | 4.7 ± 1.7 | 7.9 ± 1.4 | 9.6 ± 1.6 | 9.4 ± 2.2 | 8.9 ± 0.8 |
| | | xylose | | | (0.17 ± 0.01) | (1.01 ± 0.31) | (2.54 ± 0.35) | (4.91 ± 0.79) | (6.49 ± 0.82) | (9.48 ± 1.23) |
| pHCMC05 | P_{spac} | 0.1 mM | bgaB | 1012 | 0.0 ± 0.0 | 2.5 ± 0.4 | 2.6 ± 0.1 | 3.5 ± 0.7 | 4.2 ± 0.5 | 3.9 ± 0.9 |
| | | IPTG | | | | | | | | |
| pHCMC05 | P_{spac} | 0.5 mM | bgaB | 1012 | 0.0 ± 0.6 | 2.9 ± 0.9 | 4.5 ± 1.7 | 5.9 ± 1.7 | 6.9 ± 1.7 | 6.3 ± 1.5 |
| | | IPTG | | | | | | | | |

Cells were grown in LB medium at 37° C. to the mid-logarithmic growth phase and then induced as indicated. The data given in parentheses have been obtained by growth in NAPS medium (Kim et al., 1996).

As may be taken from table 1 acid stress resulted 60 minutes after induction in a 13-fold increase of lacZ activity in B. subtilis strain 1012 and in a 7-fold increase in strain IS58. The basal activity for lacZ differs in both strands and is 19.8 enzyme units in strain 1012 and 7.7 units in strain IS58, respectively.

Using ethanol and heat stress as inducers still resulted in a prominent increase of enzymatic activity, however, the level of enzyme activity induction was comparatively lower compared to the efficacy of acid stress. More particularly, in case of ethanol strain 1012 showed an increase of enzyme activity by a factor of 3, whereas strain IS58 showed an increase in enzyme activity by a factor of 12. Heat shock resulted in an increase in enzyme activity by a factor of 3 and 7.5, respectively. From these results it can be concluded that B. subtilis strain 1012 shows the highest induction factor upon acid stress, whereas strain IS58 shows the highest induction factor upon heat stress.

### EXAMPLE 5

### Expression Characteristics of Plasmid pHCMC03 Expressing HtpG

As a further reporter gene, the gene coding for HtpG was used which can be detected by polyclonal antibodies. The cloning of the plasmid expressing HtpG which is also referred to as pHCMC03-htpG, was otherwise identical to the one reported above for lacZ and bgaB, respectively.

B. subtilis strain 1012 containing plasmid pHCMC03 control without HtpG-gene and pHCMC03-HtpG was incubated in LB medium at 37° C. until mid-log growth phase. Subsequently the cultures were split up, one part thereof being incubated without inducer, the other one being subject to treatment using different inducers. Samples were immediately taken prior to (t=0) and 60 minutes after the treatment. The cells were lysed in all samples and the proteins of an identical number of cells were separated in SDS polyacrylamide gel. Subsequently, the protein pattern was transferred to a nylon membrane and HtpG detected using a specific polyclonal antibody.

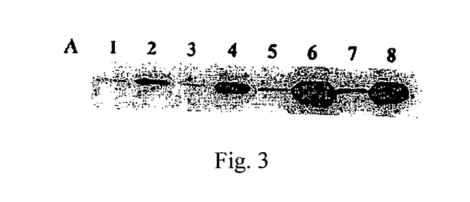

The results are depicted in FIG. 3.

As may be taken from FIG. 3 the HtpG protein could already be detected in the control strain comprising plasmid pHCMC03 without any inserted HtpG encoding gene. This is in accordance with the fact that the B. subtilis strain 1012 already contains a chromosomal HtpG gene. Upon transfection of the strain using pHCMC03-htpG the amount of HtpG was already increased compared to the level detected in insert-free pHCMC03 transfected B. subtilis 1012 (lanes 1 and 2). Upon induction of acid stress the insert-free pHCMC03 transfected cell did not show an increase of HtpG (lane 3), whereas the strain containing pHCMC03-htpG showed a significant increase in HtpG expression. Upon applying 4% ethanol (lane 6) and heat stress (lane 8), respectively, HtpG protein expression significantly increased compared to experimental conditions where acid stress was used as inducer.

Taken this, it can be concluded that in case of the expression of HtpG ethanol would be the preferred inductor.

### EXAMPLE 6

### In Vitro Expression of LTB by B. subtilis pLDV2

Both wild-type and recombinant B. subtilis strains were grown in LB in Erlenmeyer flasks aerated in an orbital shaker set at 200 rpm at 28° C. overnight. New cultures were prepared after diluting cells (1:100) in fresh medium kept at 28° C. under aeration until an OD_{600 nm} of 0.6-0.8 was reached. Heat-shocked cells were submitted to a temperature shirt after incubation at 45° C. for 2 h. Whole cell extracts were prepared after incubation of cells, corresponding to an OD_{600 nm} of 2.2 in lysis buffer (15% sucrose, 250 mM Tris-HCl pH 7.5, lysozyme 800 □g/ml) for 5 min followed by addition of 10 □l of 10% SDS and incubation at 37° C. for 15 min. SDS-PAGE was performed following standard procedures in a Mini Protean II vertical electrophoresis unit (Bio-Rad). Samples were boiled in an equal amount of sample buffer (0.625 M Tris pH 6.8, 10% v/v glycerol, 2% w/v SDS, 5% mercaptoethanol in distilled water) for 5 min and applied to 15% w/v acrylamide gels. Gels were run at 120 V and the sorted proteins were transferred to nitrocellulose sheets (0.45 μm pore size, Sigma) at 200 mA for 1 h using buffer conditions previously described (Alves, A M B; Vaccine (1998) 16:9-15). After overnight blocking with 1% w/v bovine serum albumin (BSA) in phosphate-buffered saline (PBS) at 4° C., the sheets were incubated at room temperature for 1 h with anti-LT specific polyclonal serum followed, after 3 washing steps, by incubation with 1:3,000 PBS-diluted rabbit anti-mouse IgG conjugated to horseradish peroxidase (Sigma). Membranes were developed with a chemoluminescence kit (Super Signal, Pierce), as specified by the manufacturer, and exposed to Kodak X-Omat films for 1-5 min.

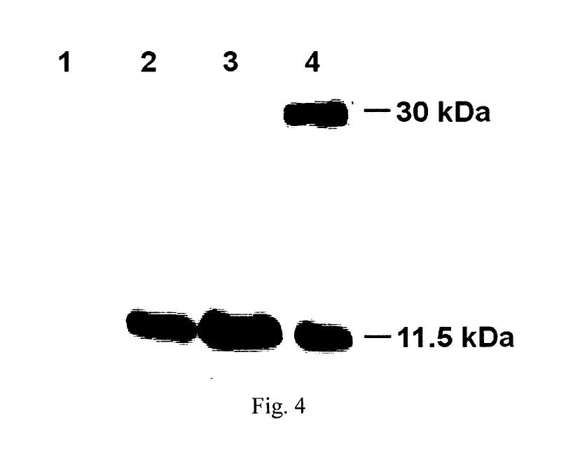

The result is shown in FIG. 4. A total of 20 μg of whole extract protein was applied per lane. The samples were as follows:

sample 1: B. subtilis LDV1 cultivated at 45° C. for 2 h;

sample 2: B. subtilis LDV2 cultivated at 28° C. up to the end of the exponential phase (culture OD 600 nm 1.4);

sample 3: B. subtilis LDV2 incubated at 45° C. for 2H (culture OD 600 nm 2.2);

sample 4: purified LT isolated from ETEC H 10407 strain;

the positions and molecular weights of LTA (30 kDa) and LTB (11.5 kDa) subunits are indicated on the right side of the figure.

As shown in FIG. 4, LTB expression was higher in cells submitted to heat-stress, but antigen expression also occurred in cells incubated at 28° C., representing approximately one fifth of the protein produced in heat-stressed cells, probably reflecting the activation of the gsiB promoter during the onset of the stationary phase due to nutrient starvation and reduced pH (Price C W; in Sonenshein A L et al., ASM press, Washington (2002) 369-84). The amount of LTB expressed by pLDV2-transformed B. subtilis cells was calculated in immunoblots as 50 ng per 10⁸ cells after the temperature increase, thus corresponding to 15 μg of antigen per dose administered via the p.o. route or 1 μg of antigen per dose administered via the i.p. route. No recombinant protein was detected in spores prepared from cultures incubated either at 28° C. or 45° C. (data not shown).

### EXAMPLE 7

### Determination of Plasmid Stability in B. subtilis Under In Vitro and In Vivo Conditions

Segregational stability was evaluated under in vitro growth conditions in B. subtilis LDV2 and LDV3 harboring vectors pLDV2 and pREP9, respectively. In vitro analysis was performed on cells grown overnight in LB medium without adding antibiotics at 37° C. under aeration in an orbital shaker (150 rpm). New cultures were prepared by dilution of overnight grown cells in approximately 1,000 CFU/ml. The procedure was repeated during a period of 7 days corresponding to approximately 225 generations. Plasmid-less colonies were detected after replica-plating neomycin-resistant colonies (the neo gene is located within the chromosome) on agar plates containing chloramphenicol (5 μg/ml), the antibiotic resistance marker encoded by pLDV2 and pREP9. All neomycin-resistant chloramphenicol-sensitive colonies were considered cured of the tested plasmid, and as much as 200 colonies were tested per interval. Sets of 10 chloramphenicol resistant colonies were also tested for LTB expression after incubation at 45° C. and Western blot analysis with whole-cell extracts.

Plasmid stability under in vivo conditions was measured in groups of 5 mice inoculated with a single p.o. dose of 10¹⁰ CFU of B. subtilis cells or spores. Groups of five female mice were kept in gridded floor cages to prevent coprophagia and fecal pellets were harvested at daily intervals for periods up to 72 h after the inoculation. Pellets collected at period were homogenized (1:10) in PBS, then, after serial dilutions in PBS, plated on DSM agar plates containing neomycin and, then, replica-plated in neomycin/chloramphenicol containing plates. In mice dosed with B. subtilis spores, fecal suspensions were incubated at 65° C. for 1 h to eliminate vegetative cells. The number of tested colonies varied from 20 to 1,500 according to the tested time points. Sets of 5 to 10 chloramphenicol resistant colonies were also submitted to Western blot experiments to evaluate LTB expression ability.

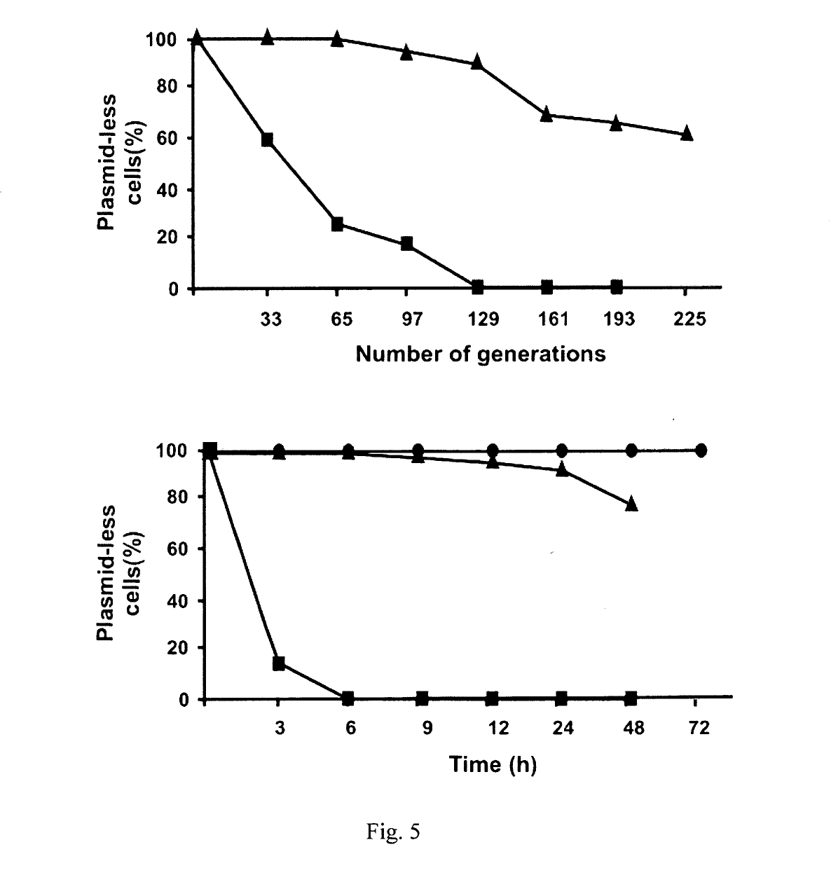

The results are indicated in FIGS. 5A and 5B. More particularly, FIG. 5A indicates the stability of pLDV2 (LDV2 strain; represented by triangles) and pREP9 (LDV3 strain, represented by squares) in B. subtilis after growth in LB at 37° C. without addition of chloramphenicol. Samples were harvested every 24 h (representing 33 generations) and plated on neomycin-containing medium followed by replica plating on chloramphenicol-containing plates. FIG. 5B indicates the stability of pLDV2 (represented by triangles and circles) and pREP9 (represented by squares) in B. subtilis cells recovered from feces of mice inoculated with a single dose of 1010 spores (represented by circles) or 3×10¹⁰ vegetative cells (represented by triangles and squares). The number of CM^{r} colonies were expressed as percentages of the total number of tested colonies.

As indicated in FIG. 5A, no segregation of pLDV2 was detected during the first 90 generations. At the end of the observation period, corresponding to 225 generations, 60% of the tested colonies still harbored the recombinant plasmid. Moreover, all clones from a subset of colonies selected by antibiotic resistance marker were able to synthesize the heterologous antigen, as evaluated by Western blots (data not shown). In comparison, another B. subtilis expression vector, pREP9, reported to replicate via a single-stranded DNA intermediate was tested. As shown in FIG. 5A, after 129 generations in LB at 37° C. all tested colonies bad lost the resistance marker encoded by the expression vector.

In vivo experiments were carried out with mice orally inoculated with a single dose of bacteria or spores, and the presence of B. subtilis cells was monitored in feces for periods of 48 h and 72 h, respectively. All of the colonies recovered from feces of mice inoculated with vegetative cells harbored the plasmid during the first 12 h, while 80% of the cells detected 48 h after the oral dosing maintained the pLDV2 expression vector (FIG. 5A). Similar to the in vitro analysis, a set of colonies carrying the pLDV2 vector was tested in immunoblots and shown to be proficient in LTB expression (data not shown). On the other hand, 100% of the cells isolated from feces of animals inoculated with B. subtilis LDV2 spores retained the expression vector throughout the observation period (FIG. 5B). For comparison, the in vivo stability of pREP9 under in vivo conditions was also evaluated. All cells recovered from feces harvested 6 h after administration of vegetative cells had lost the chloramphenicol resistance marker (FIG. 5B). These results confirmed that pLDV2 replicates stably under in vitro and in vivo conditions, thus allowing for stable antigen expression by transformed B. subtilis cells.

### EXAMPLE 8

### Immunisation of Mice Using a Vaccine Comprising B. subtilis Containing Plasmid pHCMC03 Expressing LTB Antigen

Using plasmid pHCMC03 as described in the previous examples, the LTB antigen was cloned into the plasmid pHCMC03 under the control of the gsiB promoter.

More specifically, the gsiB gene upstream region encompassing the promoter and ribosome-binding site was amplified with primers ON3 (5′ GGC CAT GGA TCC CTA TCG AGA CAC GTT TGG CTG 3′ SEQ ID NO: 4) and ON4 (5′GGC CAT GAG CTC TTC CTC CTT TAA TTG GTG TTG GT 3′, SEQ ID NO: 5, restriction sites underlined) and cloned into SacI-BamHI double-digested pMTLBS72 (Titok M A, et al., Plasmid (2003) 49:53-62). After restriction analysis, one clone containing the insert was chosen and the recombinant plasmid pLMTLgsiB isolated for a final cloning step with an amplified fragment containing the elTB gene derived from ETEC H10407 strain (Evans D G et al., Infect Immun (1975) 12:657-67). Amplification of the eltB was carried out with primers ELTBFw (5′ TCT ATG TAG ATC TAT GGC TCC TCA GTC TAT TAC AGA 3′ SEQ ID NO: 6) and ELTB2Rv (5′ TTT TAA TTC TAG ATT AGT TTT CCA TAC TGA TTG CCG C 3′ SEQ ID NO: 7). The amplified fragment was forced cloned into the BamHI and XbaI sites of pLDV1 originating in the final vector pLDV2. The correct cloning of the eltB gene was confirmed both by restriction analysis and nucleotide sequencing.

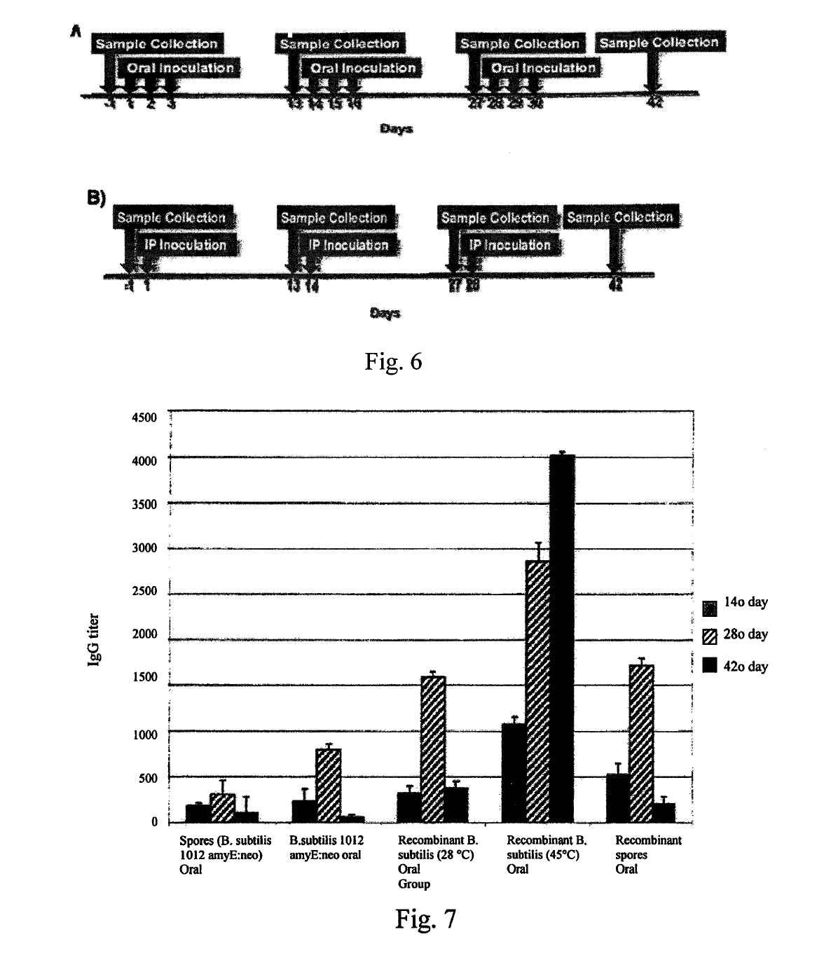

The respective plasmid, referred to herein as pHCMC03-ltb, was transfected into B. subtilis. The thus obtained B. subtilis was used either as a live vaccine, i.e. comprising vegetative cells, or as spores thereof for either intraperitoneal administration or oral administration for vaccination purposes. The two basic regimens of administration of the vaccines are indicated in FIG. 6A and FIG. 6B, whereby FIG. 6A shows the oral administration scheme, whereas FIG. 6B shows the intraperitoneal administration scheme.

For intraperitoneal administration 2×10⁹ vegetative cells or 10⁹ spores are used; for oral administration 3×10¹⁰ vegetative cells or 1.5×10¹⁰ spores are used which are administered by means of a gavage. About 50 ng of LTB protein are produced by 10¹⁰ vegetative cells.

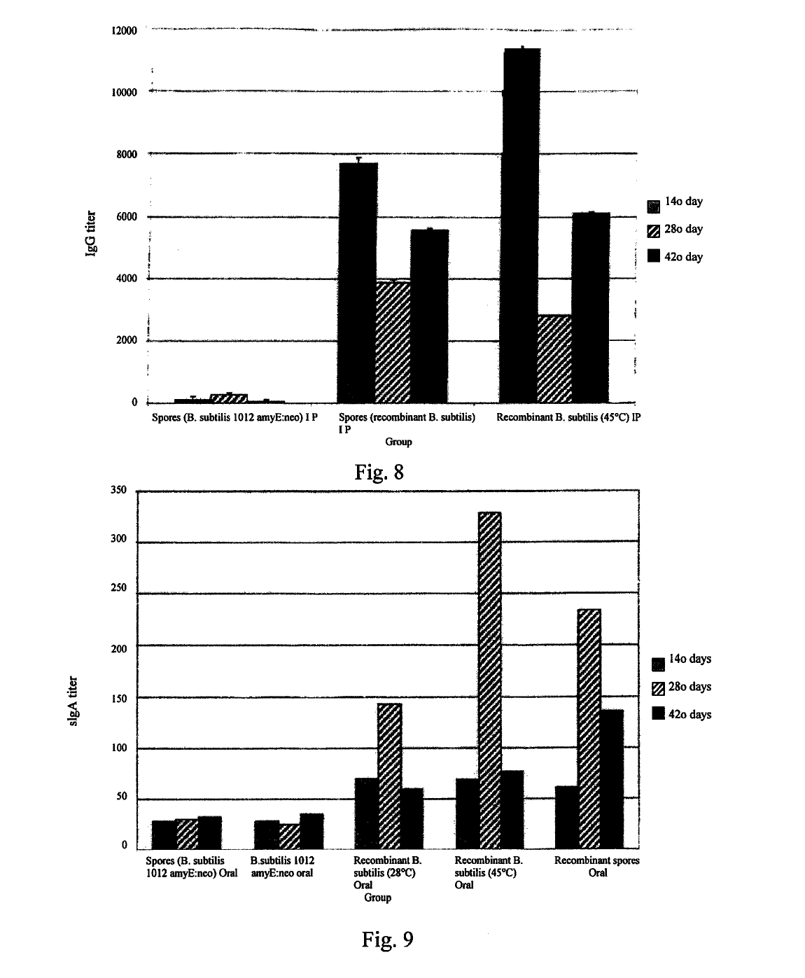

The results of the experiments are depicted in FIGS. 7 to 9. For the various groups of animals and administration regimens, samples were collected on days 14, 28 and 42 after the initial administration of the various vaccines. As may be taken from FIG. 4 both B. subtilis 1012 being either administered to the mice as spores or as vegetative cells only created a comparatively low immune response expressed as IgG titre. Both the vegetative cells as well as the spores did not contain any LTB gene but the insert-free pHCMC03 plasmid.

It may also be seen that for the LTB produced at 28° C. a significant increase in the IgG titre could be observed after 28 days. The maximum efficacy of using this vaccine can be observed, as may be taken from the fourth three columns of FIG. 7, by inducing the cells for two hours at 45° C. This resulted in a heat shock induced production of LTB which created an IgG titre increasing from day 14 to day 42. Using spores of the respective B. subtilis as vaccine still proves superior to the use of the various controls.

### EXAMPLE 9

### Serum Anti-LTB Antibody Responses Elicited in Mice Immunized with Recombinant B. subtilis Bacteria or Spores Via Parenteral and Mucosal Routes

Immunization Regimens

C57BL/6 female mice were supplied by the Isogenic Mouse Breeding Facility of the Department of Immunology, Biomedical Sciences Institute (ICB), University of Sao Paulo (USP), and all procedures were in accordance with the principles of the Brazilian code for the use of laboratory animals. Groups of five 8 weeks old female mice were inoculated per oral (p.o.) or intraperitoneally (i.p.) with vegetative cells or spores of the B. subtilis strains transformed with pLDV1 or pLDV2. P.o. immunizations were carried out with 0.5 ml aliquots of bacterial suspensions containing approximately 3×10¹⁰ CFU of vegetative cells or 1.5×10¹⁰ spores using a stainless-steel round tip gavage cannule. Mice submitted to the p.o. immunizations received 0.5 ml of in 0.1 M sodium bicarbonate 30 min before the administration of the bacterial or spore vehicles. The parenteral immunizations were performed with 2×10⁹ CFU of vegetative cells or 10⁹ spores suspended in PBS in a final volume of 0.2 ml. The immunization regimens for both cells and spores were based on previously reported attempts to use B. subtilis as vaccine vehicles (Duc L H et al.; Infect Immun (2003) 71:2810-8; Mauriello E M F et al.; Vaccine (2004) 22:1177-87). Mice immunized via the p.o. route received either three daily doses or three sets of three consecutive doses on days 1-3, 14-16, and 28-30. The i.p. immunizations were administered on days 1, 14, and 28. Serum samples were collected by puncturing the retro-orbital plexus, while feces samples were collected overnight on days −1, 13, 27 and 42. Individual blood samples of each mice group were tested for anti-LTB antibody response, pooled, and then stored at −20° C. for further ELISA tests. Fecal materials were first freeze-dried and, then, stored at −20° C. Before testing 15 fecal pellets (approximate 0.6 grams) were homogenized in 500 μl of PBS and centrifuged at 10,000 g for 10 min at 4° C. The supernatants were collected and pooled for determination of LTB-specific IgA titers.

Detection of Antigen-Specific Serum and Mucosal Antibody Responses

Detection of anti-LTB antibody responses was performed in 96-well MaxiSorp (Nunc) ELISA plates coated with 100 μl of the GM1 ganglioside (2 μg/ml in PBS buffer) per well and left at 25° C. for 4 h. Purified LT toxin (0.5 μg per well) was added to the plates and incubated for 2 h. After a blocking step with 0.1% BSA in PBS buffer for 1.5 h at 37° C., the plates were incubated for 1 h with serially diluted mouse sera or fecal extracts diluted in PBS buffer containing 0.1% BSA plus 0.05% TWEEN-20 (polyoxyethylene (20) sorbitan monolaurate). After a second washing step plates were incubated with diluted peroxidase-conjugated rabbit anti-mouse IgG or IgA (Sigma) for 1.5 h at 37° C. The plates were developed with O-phenylenediamine (0.4 mg/ml; Sigma) and H₂O₂ and reactions were stopped by adding 2 M H₂SO₄. Absorbance at 492 nm was measured on a microtiter plate reader (LabSystem). All tested samples were assayed in duplicated wells. Absorbance values of pre-immune sera or sera from non-immunized mice were used as reference blanks. Dilution curves were drawn for each sample and endpoint titres, represented by the means ±SE, were calculated as the reciprocal values of the last dilution with an optical density of 0.1.

Statistical Analysis

Antibody titres and standard deviations were calculated with the Microcal Origin 6.0 Professional program. The Student t test was applied in comparisons of mean antibody titer values of different mouse groups. Differences with P values below 0.05 were considered statistically significant.

The immunogenicity of the LDV2 strain was evaluated in mice after i.p. or p.o. inoculations of C57BUc mice with a single dose of either vegetative cells or spores, and the antibody responses were measured two weeks later.

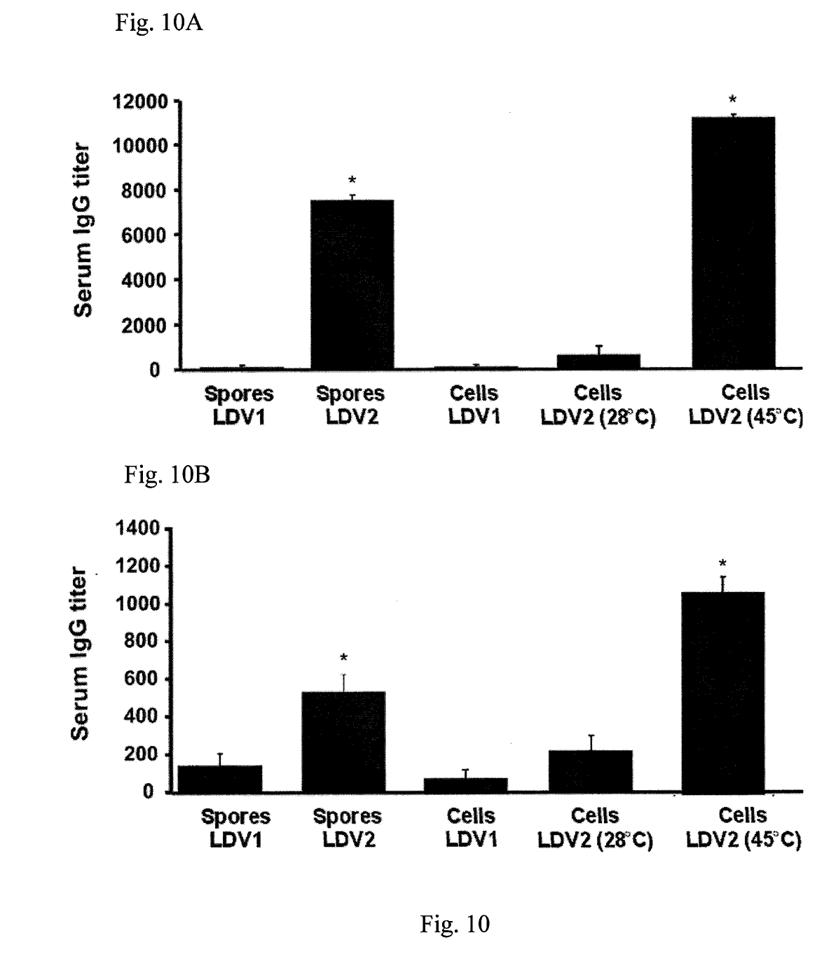

The results are shown in FIG. 10. More particularly FIG. 10A shows the immune response of mice immunized via the i.p. route with a single dose of spores (10⁹ CFU) or vegetative cells (2×10⁹ CFU) of the B. subtilis LDV1 or LDV2 strains. FIG. 10 B shows the immune response of mice immunized via the p.o. route with three consecutive daily doses of spores 1.5×10¹⁰ CFU) or vegetative cells (3×10¹⁰ CFU) of the B. subtilis LDV1 or LDV2 strains. The LDV2 strain was previously incubated at 28° C. until onset of stationary phase or heat shocked at 45° C. Blood samples were harvested 2 weeks after the last immunization. End-point titres were calculated as the reverse values of the last dilution with an optical density of 0.1.

As shown in FIG. 10, mice i.p. immunized with a single dose of 2×10⁹ live B. subtilis vegetative cells incubated at 45° C. developed anti-LT serum IgG responses (IgG titer of approximately 1.1×10⁴). Most importantly, mice immunized with a single dose of spores derived from the B. subtilis LDV2 strain also developed a specific serum antibody response (anti-LTB titer of 7.7×10³), as compared to mice immunized with non-recombinant spores, suggesting that LTB expression bad occurred during in vivo conditions, probably after spore germination during transit through the gastrointestinal tract and/or in phagolysosomes of phagocytic cells.

Analysis of the anti-LTB serum IgG responses in mice p.o. inoculated with three daily doses of bacteria or spores of the LDV 1 strain confirmed that, similar to parenterally immunized mice, serum anti-LTB IgG responses were induced both with vegetative cells incubated at 45° C. (average IgG titre of 1.1×10³) and spores (average IgG titre of 5×10²), when compared to the responses elicited in mice inoculated with the non-recombinant strain either as vegetative cells or spores (FIG. 10). In contrast to cells incubated at 45° C., no statistically significant IgG response was detected in mice orally inoculated with B. subtilis cells cultivated at 28° C.

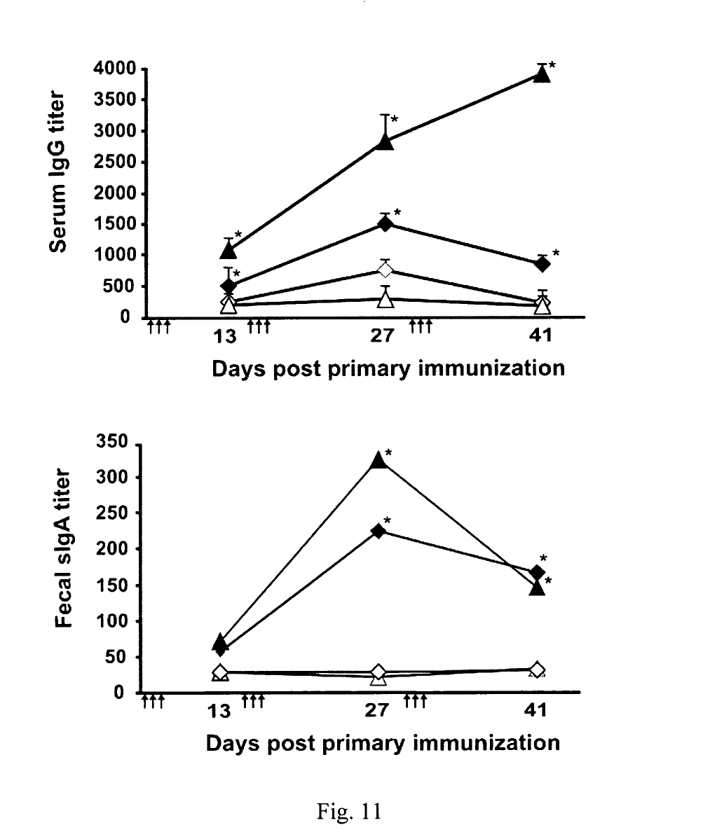

Previously tested oral immunization regimens based on recombinant B. subtilis strains as live vaccine vectors employed three daily consecutive injections repeated three times at intervals of 2 weeks each, a procedure adapted to counterbalance the low immunogenicity of B. subtilis spores and vegetative cells (Duc L H et al.; Infect Immun (2003) 71:2810-8; Mauriello E M F et al.; Vaccine (2004) 22:1177-87). The same immunization regimen was repeated employing vegetative cells or spores of the B. subtilis LDV2 strain. The results are shown in FIG. 11.

As shown in FIG. 11, the induced serum IgG responses varied according to the antigen vehicle used. Mice orally immunized with vegetative cells incubated at 45° C. induced increased LTB-specific serum antibody responses with maximal values achieved two weeks after the last dose (serum IgG titre of 4×10³). On the other hand, mice immunized with B. subtilis spores developed peak anti-LTB responses 2 weeks after the second immunization dose (serum IgG titer of 1.5×10³) and, then, showed a decrease in the LTB-specific serum IgG response after the third set of doses (FIG. 10).

### EXAMPLE 10

### Anti-LTB Secreted Antibody Responses Elicited in Mice Immunized with the Recombinant B. subtilis LDV2 Strain

Similar to the serum IgG responses, all mice p.o. immunized with vegetative cells or spores elicited LTB-specific fecal IgA responses with peak antibody responses detected after the second set of doses (average IgA titres of 2.3×10² and 3.3×10² for mice immunized with spores or vegetative cells, respectively) as represented in FIG. 11. After the third set of doses, the specific anti-LTB IgA response levels clearly dropped with a final IgA titre corresponding to one-half and one-third of those detected in mice inoculated with two doses of spores or vegetative cells, respectively (FIG. 11).

More particularly, FIG. 11 depicts the induction of LTB-specific serum IgG (A) and fecal IhA (B) responses elicited in mice p.o. immunized with spores or vegetative cells of B. subtilis LDV1 and LDV2 strains. Groups of five mice were immunized with three series of three consecutive daily doses containing 3×10¹⁰ CFU of vegetative cells or 1.5×10¹⁰ spores of the recombinant B. subtilis LDV2 strain of the LDV1 strain. The tested samples were as follows:

B. subtilis LDV1 spores (represented by open circles);

B. subtilis LDV1 vegetative cells (represented by open triangles);

B. subtilis LDV2 spores (represented by closed circles); and

B. subtilis LDV2 vegetative cells (represented by closed triangles).

The arrows indicate the days of each immunization. End-point titres were calculated as reverse values of the last dilution with an optical density of 0.1. * Statistically significant differences (P<0.005) with regard to respective samples collected from mpuce groups immunized with the LDV1 strain.

### EXAMPLE 11

### Analysis of IgG Subclasses

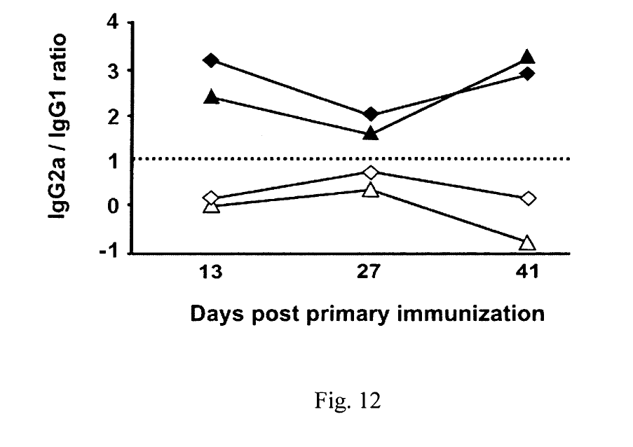

The LTB-specific IgG subclass (IgG1 and IgG2a) responses elicited in mice immunized with recombinant B. subtilis spores and vegetative cells were measured in serum samples collected from animals inoculated via i.p. or p.o. immunizations routes. As indicated in FIG. 12, mice immunized via the p.o. route, both with spores or vegetative cells incubated at 45° C., developed a prevailing type 1 response as indicated by the predominant IgG2a subclass responses, particularly among mice immunized with recombinant spores. On the other hand, the immune responses elicited in mice immunized via the i.p. route tended to express a type 2 response with a predominant IgG1 subclass response both with spores or vegetative cells (FIG. 12).

More particularly, FIG. 12 shows the serum anti-LTB IgG subclasses response elicited in mice immunized with spores or vegetative cells of the B. subtilis LDV2 strain via i.p. or p.o. routes. Serum samples were harvested on different days post immunization from mice immunized via the i.p. route (open symbols) or p.o. route (filled symbols) with spores (circles) or vegetative cells (triangles) of the B. subtilis LDV2 strain. Mice immunized via the i.p. route received three doses at days 1, 14, and 28 while mice submitted to the p.o. immunization regimen received 3 doses at days 1-3, 14-16, and 28-30. The ratio of IgG2a/IgG1 for each tested serum sample is indicated according to the time schedule of the immunization regimens.

The features of the present invention disclosed in the specification, the claims and/or the drawings may both separately and in any combination thereof be material for realizing the invention in various forms thereof.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for expressing a nucleic acid comprising using an expression system comprising a gsiB promoter as an inducible promoter, whereby the promoter can be induced by a measure selected from the group consisting of decrease in pH, increase in temperature, addition of alcohol, exhaustion of nutrients, and oxygen limitation.

2. The method according to claim 1, wherein the decrease in pH is a decrease in pH of the culture medium.

3. The method according to claim 2, wherein the decrease in pH is from about 6.8 to about 5.8.

4. The method according to claim 1, wherein the increase in temperature is an increase of about at least 10° C., preferably from about 37° C. to about 48° C.

5. The method according to claim 1, wherein the alcohol is ethanol and the addition of alcohol results in an ethanol level within the culture medium of about 4%.

6. The method according to claim 1, wherein the promoter comprises a sequence according to SEQ. ID. NO: 1.

7. The method according to claim 1, wherein the gsiB promoter is incorporated into an expression vector.

8. The method according to claim 1, wherein the expression system further comprises a microorganism of the genus Bacillus.

9. The method according to claim 1, wherein the expression system is for the production of a polypeptide.

10. The method according to claim 8, wherein the expression system is for the use as a vaccine.

11. A nucleic acid replicon that replicates in Bacillus, for expression of a polypeptide, whereby the replicon comprises a gsiB promoter and a plasmid selected from the group consisting of pMTLBS72, pAMβ1, and pTB19.

12. The nucleic acid replicon according to claim 11, wherein the gsiB promoter is inserted into a SacI-BamHI restriction site.

13. The nucleic acid replicon according to claim 11, wherein the replicon comprises a transcriptional terminator.

14. The nucleic acid replicon according to claim 13, wherein the transcriptional terminator is selected from the group consisting of a trpA transcriptional terminator, a to terminator of bacteriophage lambda, and a t₁t₂ terminator of a rrnB operon.

15. The nucleic acid replicon according to claim 13, wherein the transcriptional terminator is inserted between a MluI and an AatII restriction site of pMTLBS72.

16. The nucleic acid replicon according to claim 13, wherein the promoter and the transcriptional terminator form an expression cassette.

17. The nucleic acid replicon according to claim 16, wherein the expression cassette is inserted between a pair of restriction sites of pMTLBS72, whereby such pair of restriction sites is selected from the group consisting of SacI-BamHI, SacI-XbaI, SacI-AatII, BamHI-XbaI, BamHI-AatII, and XbaI-AatII.

18. The nucleic acid replicon according to claim 11, wherein the replicon further comprises at least one element selected from the group consisting of an origin, and a selection marker.

19. The nucleic acid replicon according to claim 11, wherein the replicon comprises a nucleic acid sequence coding for a polypeptide, whereby the gsiB promoter controls expression of the polypeptide.

20. The nucleic acid replicon according to claim 11, wherein the polypeptide is selected from the group consisting of enzymes, pharmaceutically active polypeptides, and antigens.

21. The nucleic acid replicon according to claim 20, wherein the polypeptide is a β subunit of heat labile toxin B (LTB) antigen.

22. The nucleic acid replicon according to claim 11, wherein the replicon is a vector.

23. The nucleic acid replicon according to claim 22, wherein the vector is a shuttle vector for both E. coli and B. subtilis.

24. A host cell comprising a nucleic acid replicon according to claim 11.

25. The host cell according to claim 24, wherein the host cell is selected from genus Bacillus.

26. The host cell according to claim 25, wherein the host cell is Bacillus subtilis.

27. The host cell according to claim 26, wherein the host cell is selected from the group consisting of Bacillus subtilis strain 1012 and Bacillus subtilis strain IS58.

28. The host cell according to claim 24, wherein the host cell is E. coli.

29. A vaccine comprising a host cell according to claim 24, wherein the host cell is Bacillus.

30. The vaccine according to claim 29, wherein the host cell is Bacillus subtilis.

31. The vaccine according to claim 29, wherein the host cell is selected from the group consisting of Bacillus subtilis strain 1012 and Bacillus subtilis strain IS58.

32. The vaccine according to claim 29, wherein the vaccine is an oral vaccine.

33. The vaccine according to claim 29, wherein the vaccine elicits a specific immune response.

34. The vaccine according to claim 29, wherein the vaccine comprises vegetative Bacillus.

35. The vaccine according to claim 29, wherein the vaccine comprises Bacillus spores.

36. The vaccine according to claim 29, wherein the antigen expressed by the host cell is LTB antigen.

37. The vaccine according to claim 29 for the treatment of a subject, whereby the subject is an animal and/or a human being.

38. The vaccine according to claim 37, wherein the animal is a domestic animal selected from the group consisting of cattle, sheep, pigs, goats, horses, dogs, cats, and birds.

39. The vaccine according to claim 29, wherein the polypeptide expressed by the host cell is LTB and the vaccine is for the treatment of LTB associated diarrhoea.

40. The vaccine according to claim 29, wherein the vaccine is for treatment and/or prevention of a disease.

41. A method for producing a polypeptide comprising the steps of:
d) providing a host cell according to claim 24, whereby the host cell encodes for the polypeptide;
e) cultivating the host cell under conditions allowing for the expression of the polypeptide; and
f) harvesting the polypeptide.

42. A method for providing an immune response in a subject comprising the steps of:
c) providing a vaccine according to claim 29; and
d) administering the vaccine to the subject in an amount so as to elicit an immune response.

43. The method according to claim 42, wherein the subject is a human being or an animal.


SEQUENCE LISTING (8 sequences)

SEQ ID NO: 1 - DNA, 61 bp, Bacillus subtilis
  promoter (1)..(61)
  promoter (1)..(61): Position  5 - 29 promoter sequence Position 40 - 55 non-translated stretch Position 40 translation start Position 49 - 55 SD sequence
gtttgtttaaaagaattgtgagcgggaatacaacaaccaacaccaattaaaggaggaattc

SEQ ID NO: 2 - DNA, 39 bp, Artificial
  linker
ggccatggatcctcactcctactattaaacgcaaaatac

SEQ ID NO: 3 - DNA, 39 bp, Artificial; identical to SEQ ID NO: 2
  linker

SEQ ID NO: 4 - DNA, 33 bp, Artificial
  primer
ggccatggatccctatcgagacacgtttggctg

SEQ ID NO: 5 - DNA, 35 bp, Artificial
  primer
ggccatgagctcttcctcctttaattggtgttggt

SEQ ID NO: 6 - DNA, 36 bp, Artificial
  primer
tctatgtagatctatggctcctcagtctattacaga

SEQ ID NO: 7 - DNA, 37 bp, Artificial
  primer
ttttaattctagattagttttccatactgattgccgc

SEQ ID NO: 8 - protein, 123 aa, Bacillus subtilis
MADNNKMSREEAGRKGGETTSKNHDKEFYQEIGQKGGEATSKNHDKEFYQEIGEKGGEATSKNHDKEFYQEIGEKGGEATSENHDKEFYQEIGRKGGEATSKNHDKEFYQEIGSKGGNARNND